(19) United States
(12) Patent Application Publication
(10) Pub. No.: US 2025/0235266 A1
(43) Pub. Date: Jul. 24, 2025

(54) METHODS AND APPARATUS FOR EVALUATING POSITION STABILITY OF A MEDICAL DEVICE

(71) Applicant: Abiomed, Inc., Danvers, MA (US)

(72) Inventors: Qingchao Kong, Danvers, MA (US); Alexander Ship, Danvers, MA (US); Daniel Raess, Danvers, MA (US)

(21) Appl. No.: 19/034,110

(22) Filed: Jan. 22, 2025

Related U.S. Application Data
(60) Provisional application No. 63/623,644, filed on Jan. 22, 2024.

(51) Int. Cl. A61B 34/10; A61B 5/11; A61B 90/00

(52) U.S. Cl. CPC A61B 34/10; A61B 5/1127; A61B 5/1128; A61B 90/39; A61B 2034/107; A61B 2090/3908; A61B 2090/3966

(57) ABSTRACT

Disclosed are devices, systems, and techniques for evaluating position support for a medical device. The method includes performing a movement evaluation of one or more extremities of a patient associated with the medical device, determining, using at least one computer processor, passive movement of one or more muscles of the patient during the movement evaluation, and determining a fixation plan for fixing at least a portion of the medical device to one or more anatomical tissues of the patient based, at least in part, on the passive movement of the one or more muscles.

### RELATED APPLICATIONS

This application claims the benefit under 35 U.S.C. § 119 (e) to U.S. Provisional Patent Application No. 63/623,644, filed Jan. 22, 2024, and titled, “METHODS AND APPARATUS FOR EVALUATING POSITION STABILITY OF A MEDICAL DEVICE,” the entire contents of which is incorporated by reference herein.

### TECHNICAL FIELD

The present disclosure relates to techniques for evaluating the position stability of medical devices.

### BACKGROUND

In some instances, a medical device, such as a blood pump, may need to be introduced and placed within a patient. During usage of the medical device the patient may be mobile, which may result in the position of the medical device moving from the location where it was initially placed. One or more portions of the medical device may be fixated to the anatomy of the patient to limit movement of the medical device.

### SUMMARY

In some embodiments, a method for evaluating position support for a medical device is provided. The method includes performing a movement evaluation of one or more extremities of a patient associated with the medical device, determining, using at least one computer processor, passive movement of one or more muscles of the patient during the movement evaluation, and determining a fixation plan for fixing at least a portion of the medical device to one or more anatomical tissues of the patient based, at least in part, on the passive movement of the one or more muscles.

In one aspect, performing the movement evaluation comprises performing a position evaluation and/or rotation evaluation of one or more joints and/or muscles associated with the one or more extremities of the patient. In another aspect, the one or more extremities includes one or more upper extremities of the patient. In another aspect, the one or more extremities includes one or more lower extremities of the patient. In another aspect, performing a movement evaluation of one or more extremities of the patient comprises tracking a location of one or more markers located on and/or within the patient as the one or more extremities are moved through one or more trajectories, and determining passive movement of one or more muscles of the patient comprises determining passive movement of the one or more muscles based, at least in part, on the tracked location of the one or more markers. In another aspect, at least one of the one or more markers is located on the medical device. In another aspect, tracking a location of one or more markers comprises performing imaging of the patient as the one or more extremities are moved through the one or more trajectories to generate a plurality of images, the plurality of images including representations of at least some of the one or more markers. In another aspect, at least some of the one or more markers are radiopaque markers. In another aspect, performing imaging of the patient comprises performing fluoroscopic imaging. In another aspect, the one or more markers are located subcutaneously within the patient.

In another aspect, determining a fixation plan for fixing at least a portion of the medical device to the one or more anatomical tissues of the patient comprises determining a number of fixation points to use to fix the at least a portion of the medical device to the one or more anatomical tissues of the patient. In another aspect, determining a fixation plan for fixing at least a portion of the medical device to the one or more anatomical tissues of the patient comprises determining which of the one or more anatomical tissues of the patient to fix the at least a portion of the medical device. In another aspect, determining a fixation plan for fixing at least a portion of the medical device to the one or more anatomical tissues of the patient comprises determining a fixation plan that minimizes predicted movement of the medical device in the patient as the patient moves during usage of the medical device.

In another aspect, the method further includes fixing the at least a portion of the medical device to the one or more anatomical tissues of the patient based on the fixation plan. In another aspect, the method further includes monitoring a position stability of the medical device as the patient ambulates during usage of the medical device, and determining an updated fixation plan based, at least in part, on the monitoring. In another aspect, the medical device is implanted in the patient, and performing a movement evaluation of one or more extremities of a patient associated with the medical device is performed as the patient ambulates during usage of the medical device. In another aspect, the medical device is an implantable heart pump.

In another aspect, the medical device includes a catheter coupled between a first portion of the medical device configured to be implanted in the patient and a second portion of the medical device configured to be located outside of the patient, and determining a fixation plan comprises determining a fixation plan for fixing a portion of the catheter configured to be located inside of the patient to the one or more anatomical tissues of the patient. In another aspect, the one or more anatomical tissues of the patient includes one or more of a pectoralis minor muscle, a pectoralis major muscle, a trapezius muscle, a deltoid muscle, or an external oblique muscle.

In another aspect, the movement evaluation is performed after inserting the medical device into the patient and prior to operation of the medical device. In another aspect, the method further includes fixing the at least a portion of the medical device to the one or more anatomical tissues of the patient based on the fixation plan.

In another aspect, the fixation plan is further based, at least in part, on information about how the patient may move during usage of the medical device. In another aspect, determining a fixation plan for fixing at least a portion of the medical device to one or more anatomical tissues of the patient based, at least in part, on the passive movement of the one or more muscles comprises determining a fixation plan for fixing at least a portion of the medical device to the one or more muscles. In another aspect, the fixation plan is further based, at least in part, on information about how the patient may move during usage of the medical device. In another aspect, determining a fixation plan for fixing at least a portion of the medical device to one or more anatomical tissues of the patient based, at least in part, on the passive movement of the one or more muscles comprises determining a fixation plan for fixing at least a portion of the medical device to the one or more muscles.

In some embodiments, a position support evaluation system for a medical device is provided. The system includes at least one computer processor programmed with a plurality of processor executable instructions that, when executed by the at least one computer processor perform a method. The method includes determining passive movement of one or more muscles of a patient associated with the medical device during a movement evaluation of one or more extremities of the patient, determining a fixation plan for fixing at least a portion of the medical device to one or more anatomical tissues of the patient based, at least in part, on the passive movement of the one or more muscles, and outputting an indication of the fixation plan.

In one aspect, performing the movement evaluation comprises performing a position evaluation and/or rotation evaluation of one or more joints and/or muscles associated with the one or more extremities of the patient. In another aspect, the one or more extremities includes one or more upper extremities of the patient. In another aspect, the one or more extremities includes one or more lower extremities of the patient. In another aspect, performing a movement evaluation of one or more extremities of the patient comprises tracking a location of one or more markers located on and/or within the patient as the one or more extremities are moved through one or more trajectories, and determining passive movement of one or more muscles of the patient comprises determining passive movement of the one or more muscles based, at least in part, on the tracked location of the one or more markers. In another aspect, at least one of the one or more markers is located on the medical device. In another aspect, tracking a location of one or more markers comprises performing imaging of the patient as the one or more extremities are moved through the one or more trajectories to generate a plurality of images, the plurality of images including representations of at least some of the one or more markers. In another aspect, the at least some of the one or more markers are radiopaque markers. In another aspect, performing imaging of the patient comprises performing fluoroscopic imaging. In another aspect, the one or more markers are located subcutaneously within the patient.

In another aspect, determining a fixation plan for fixing at least a portion of the medical device to the one or more anatomical tissues of the patient comprises determining a number of fixation points to use to fix the at least a portion of the medical device to the one or more anatomical tissues of the patient. In another aspect, determining a fixation plan for fixing at least a portion of the medical device to the one or more anatomical tissues of the patient comprises determining which of the one or more anatomical tissues of the patient to fix the at least a portion of the medical device. In another aspect, determining a fixation plan for fixing at least a portion of the medical device to the one or more anatomical tissues of the patient comprises determining a fixation plan that minimizes predicted movement of the medical device in the patient as the patient moves during usage of the medical device. In another aspect, the medical device is implanted in the patient, and performing a movement evaluation of one or more extremities of a patient associated with the medical device is performed as the patient ambulates during usage of the medical device. In another aspect, the medical device is an implantable heart pump.

In another aspect, the medical device includes a catheter coupled between a first portion of the medical device configured to be implanted in the patient and a second portion of the medical device configured to be located outside of the patient, and determining a fixation plan comprises determining a fixation plan for fixing a portion of the catheter configured to be located inside of the patient to the one or more anatomical tissues of the patient. In another aspect, the one or more anatomical tissues of the patient includes one or more of a pectoralis minor muscle, a pectoralis major muscle, a trapezius muscle, a deltoid muscle, or an external oblique muscle. In another aspect, the movement evaluation is performed after inserting the medical device into the patient and prior to operation of the medical device.

### BRIEF DESCRIPTION OF DRAWINGS

FIG. 1 schematically illustrates positioning of portions of a medical device in a patient, in accordance with some embodiments of the present disclosure.

FIGS. 2A-2D schematically illustrate alternate fixation plans for a medical device in accordance with some embodiments of the present disclosure.

FIG. 3 is a flowchart of a process for evaluating position stability of a medical device, in accordance with some embodiments

### DETAILED DESCRIPTION

Cardiovascular diseases are a leading cause of morbidity and mortality. A variety of treatment modalities have been developed for heart health, ranging from pharmaceuticals to mechanical devices and transplantation. Temporary cardiac support devices, such as heart pump systems, provide hemodynamic support and facilitate heart recovery. Some heart pump systems may be percutaneously inserted into the heart and can operate in parallel with the native hart to supplement cardiac output. Examples of such devices include the Impella® family of devices, Abiomed, INC., Danvers, MA).

For some treatment regimes, such as bridge-to-recovery (BTR) or bridge to transplant chronic systems for future home-discharge, the pump system may need to have a certain amount of driveline subcutaneously positioned for securement, and may need to ensure the patient has a good quality of life during such home discharge chronic support.

To limit movement of the subcutaneous driveline, or similar subcutaneously positioned tubular members, at least a portion of the subcutaneous portions of the device may be secured or “fixed” to one or more muscles in the patient's musculature at one or more fixation points.

Disclosed are various devices, systems, and techniques that enable a physician or other healthcare professional to evaluate, predict and/or monitor the position stability of a medical device (e.g., an implantable blood pump). As a non-limiting example of a medical device that may be used in accordance with some embodiments of the present disclosure, a blood pump may include a pump section coupled to a tubular member (e.g., a catheter). A sheath may be disposed over at least a portion of the pump section and a portion of the tubular member. A fixation sleeve may be disposed around a portion of the tubular member, and may contain a medically acceptable textile or fiber. The fixation sleeve may be configured to be fixed to a patient's anatomy (e.g., one or more muscles in a patient's musculature) during implantation of the device and/or subsequent to implantation to the device (e.g., if the device needs to be repositioned).

The inventors have recognized and appreciated that due, at least in part, to different anatomies of patients and/or a lack of information about how the patient may ambulate during usage of the medical device, it can be challenging for a physician to determine how to best to fix a portion of the medical device (e.g., a catheter of an implantable blood pump) to a patient's musculature in a way that will limit movement of the medical device when in operation, while permitting desired movement of the patient's extremities. To this end, some embodiments of the present disclosure relate to methods and apparatus for evaluating position stability of a medical device when fixed to patient's musculature according to a fixation plan. The fixation plan may describe how at least a portion of the medical device is fixed to the patient's anatomy at one or more fixation points associated with one or more muscles of the patient.

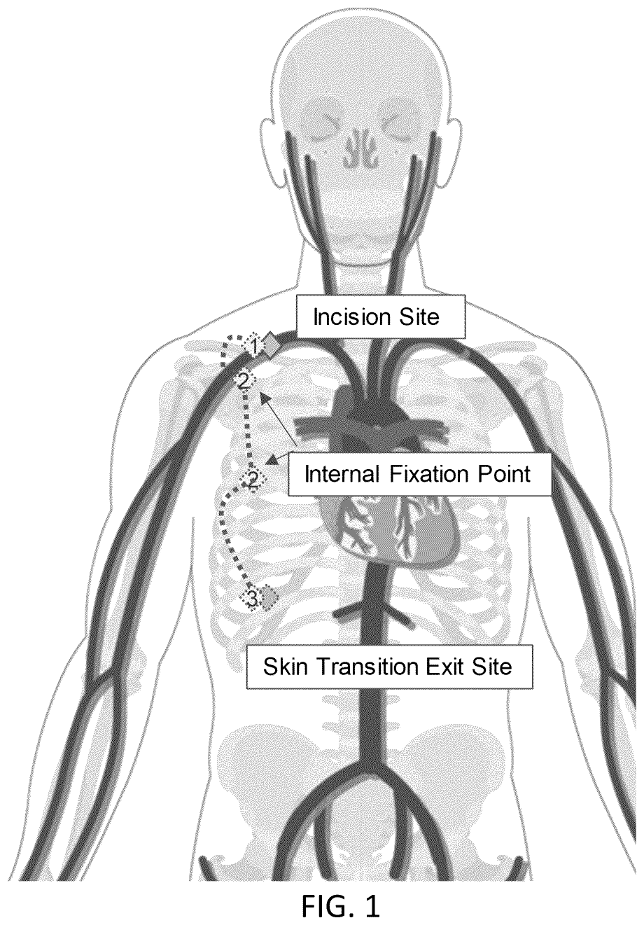

Some implantable blood pumps may be inserted transvascularly into a patient's heart via an axillary artery anastomosis, and may be subcutaneously fixated at one or more fixation points to the patient's musculature. FIG. 1 schematically illustrates the positioning of portions of an implantable blood pump, in accordance with some embodiments. In the example shown in FIG. 1, a tunnelling procedure may be used to create a tunnelling path for a tubular portion (e.g., a catheter) of the medical device (e.g., a blood pump) from an incision site to a skin transition exit site. An anastomosis created at the incision site may provide for fixation of the pump for pump internal position stability (e.g., forming a primary torsion constraint). The skin transition exit site may isolate expected external loads on the medical device and common use errors (e.g., controller drops) from the positioning of the implanted part of the medical device. The tunnelling path may include one or more internal fixation points at which the tubular portion of the medical device may be fixated to the patient's musculature after insertion of the blood pump into the tunneling path. For example, the internal fixation points may be selected to provide strain relief by isolating expected external loads on the pump from the internal pump position. In the example shown in FIG. 1, two internal fixation points are shown, though it should be appreciated that any suitable number of fixation points may be used, and embodiments of the present disclosure are not limited in this respect.

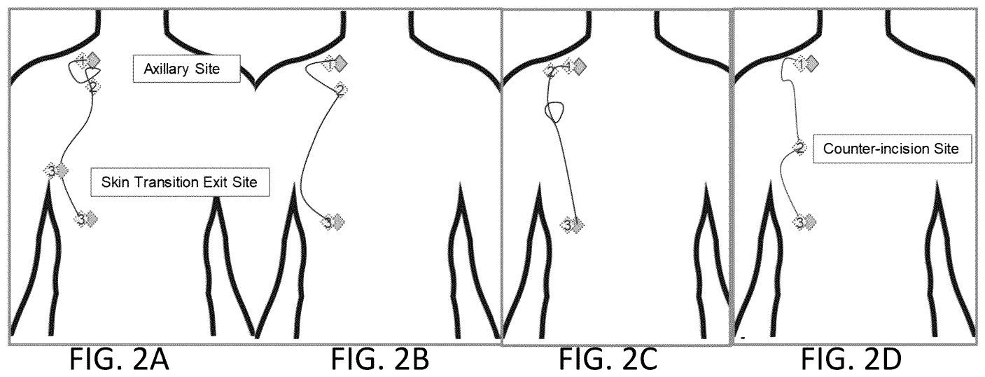

With limited information in the literature regarding relative translation of subcutaneous and intravascular anatomies relative to in-dwelling catheters during patient activity, it may be challenging to determine a fixation plan for the medical device that minimizes or otherwise reduces movement of the implanted medical device during patient movement. For instance, movement of the patient's arms following implantation of a heart pump (e.g., according to the tunneling path shown in FIG. 1) may result in movement of the implanted device. Placing and fixating the device to the patient's musculature in accordance with certain fixation plans (e.g., different tunneling paths and/or using different internal fixation point(s)) may result in more and/or undesirable types of device movement compared to other fixation plans. For example, FIGS. 2A-2D schematically illustrate alternate fixation plans for a medical device in accordance with some embodiments. As can be appreciated from the different fixation plans shown in FIGS. 2A-2D, a fixation plan may include a different tunneling path and/or a different number and/or position of internal fixation points. Accordingly, some embodiments of the present disclosure relate to techniques for determining a fixation plan that is likely to improve position stability of the implanted medical device during patient movement. In some embodiments, a particular fixation plan may be determined, at least in part, on information about expected movements of the patient's extremities during usage of the medical device.

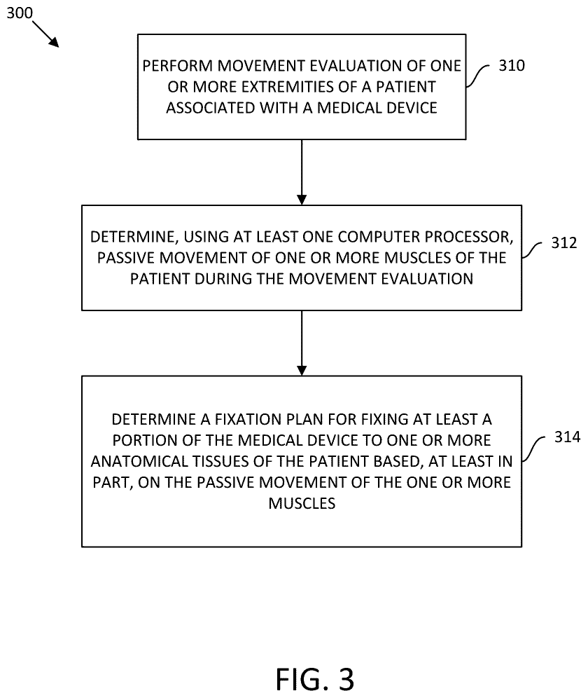

FIG. 3 is a flowchart of a process 300 for evaluating position stability of a medical device, in accordance with some embodiments. Process 300 begins in act 310, where a movement evaluation of one or more extremities of a patient is performed. In some embodiments, the movement evaluation may be performed prior to inserting the medical device in the patient (e.g., during pre-surgical planning). In other embodiments, the movement evaluation may be performed after inserting the medical device in the patient but prior to operation of the device (e.g., during surgery but prior to fixation of the device). In yet further embodiments, the movement evaluation may be performed after implantation and operation of the medical device (e.g., to determine whether the fixation plan should be updated due to the presence of too many mispositioning alarms output by the medical device). In some embodiments, the movement evaluation may be performed at multiple of the above-described times or at any other suitable time.

In some embodiments, the movement evaluation may be performed by placing one or more markers on the patient's anatomy and/or the medical device (e.g., when the movement evaluation is performed after insertion of the medical device into the patient), and performing imaging during patient movement to determine how the position of the one or more markers change in response to the patient movement. For example, the one or more markers may include one or more radiopaque markers, and imaging (e.g., fluoroscopic imaging) may be performed as one or more of the patient's extremities are moved into different positions and/or rotations. In some embodiments, the movement evaluation may include moving the patient's extremities (e.g., upper extremities) into positions and/or rotations that the patient is likely to experience or want to perform during usage of the device.

In some embodiments, the marker(s) may be placed at locations on the surface of the patient's body (e.g., on the patient's skin). In some embodiments, the marker(s) may be placed at one or more subcutaneous locations. When placed on the surface of the patient's body, performing imaging may include using a camera to detect the position of the marker(s) as the patient moves. When placed subcutaneously, performing imaging may include using a non-invasive imaging technique configured to detect the markers when placed. In some embodiments, the output of the movement evaluation may be a tracked location of the one or more markers as one or more extremities of the patient are moved through one or more trajectories. For instance, the locations of the marker(s) may be tracked as the patient's arm is raised and lowered relative to their torso, when the patient's arm is rotated at their shoulder joint, etc. In some embodiments, the patient's right arm may be manipulated through a series of positions while images are captured in Anterior-Posterior and Right Anterior Oblique views to explore the range of motion of the patient's shoulder. The locations of the marker(s) may be determined by performing 2D and/or 3D reconstructions of marker and/or medical device position.

Returning to process 300, process 300 may proceed to act 312, where passive movement of one or more muscles of the patient may be determined during the movement evaluation. For instance, the tracked locations of the marker(s) during arm movement may be used to determine the passive movement of one or more muscles in the patient's shoulder and/or chest (e.g., the pectoralis minor muscle, the pectoralis major muscle, the trapezius muscle, the deltoid muscle, the external oblique muscle, etc.).

Process 300 may then proceed to act 314, where a fixation plan for fixing at least one portion of the medical device to one or more anatomical tissues of the patient is determined. For example, the fixation plan may include a location and/or configuration of at least a portion of a tunneling path for the medical device, a location of the incision site and/or exit site of the tunneling path, a number and/or location of fixation points along the tunneling path, which muscle(s) to use (or avoid) when fixing at least a portion of the medical device, etc. In some embodiments, determining the fixation plan may include determining the fixation plan that minimizes the predicted movement of the medical device as the patient moves in various ways (e.g., various ways in which the patient is expected to move during usage of the medical device). In some embodiments, the fixation plan may be determined using one or more numerical optimization techniques with the objective to minimize undesirable device movement during patient movement.

In some embodiments, the fixation plan may further be determined based, at least in part, on information about how the patient may move during usage of the medical device. For example, if the patient is expected to have to move their arm into a particular position (e.g., to perform their job, to improve their quality of life, etc.) during usage of the medical device, that information may be taken into account when determining the fixation plan (e.g., as weights and/or other information provided to an optimization process).

In some embodiments, following determination of a fixation plan, a physician (e.g., a surgeon) may proceed to fixate at least a portion of the medical device to the patient's anatomy (e.g., one or more muscles or other appropriate anatomical tissues) based on the fixation plan. For instance, a catheter for an implantable medical device may be configured with a fixation component that can be arranged at different locations along the catheter. The location(s) of the fixation component may be selected based on the fixation plan, such that when the catheter is inserted into the patient, the fixation component may be arranged at a desired fixation location according to the fixation plan.

Although the techniques described herein may be used for pre-surgical planning and/or during surgical implantation of a medical device, the inventors have recognized and appreciated that the techniques described herein may also be used to monitor how movements of the patient during usage of the medical device result in movements of the device. For example, during operation, a medical device (e.g., a blood pump) may be configured to output an alert signal when the position of the device deviates from a desired position (e.g., the pump is not located across a particular heart valve to pump blood across the valve). Although the alerts may be helpful to inform a healthcare provider that the medical device should be repositioned to ensure proper functioning, it may be challenging to determine whether patient movement or some other factor(s) resulted in the mispositioning of the medical device. Some embodiments of the present disclosure may be used to evaluate whether patient movement is a contributing cause of the mispositioning alarms or whether other factors may be the cause. For instance, a patient may be instructed to rotate their shoulder in a particular way, and the location of one or more markers associated with the medical device may be tracked using the techniques described herein to determine how the medical device moves in response to the rotation of the patient's shoulder. If the medical device is not displaced substantially to cause a mispositioning alarm, it may be determined that factors other than patient movement may be causing the alarms.

In some embodiments, an updated fixation plan may be generated when it is determined that repositioning of the fixed portion of the medical device may be beneficial to prevent further movement of the medical device. For example, the updated fixation plan may include addition and/or alternative fixation points to fixate the medical device to the patient's anatomy to facilitate position stability of the medical device.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method for evaluating position support for a medical device, the method comprising:
performing a movement evaluation of one or more extremities of a patient associated with the medical device;
determining, using at least one computer processor, passive movement of one or more muscles of the patient during the movement evaluation; and
determining a fixation plan for fixing at least a portion of the medical device to one or more anatomical tissues of the patient based, at least in part, on the passive movement of the one or more muscles.

2. The method of claim 1, wherein performing the movement evaluation comprises performing a position evaluation and/or rotation evaluation of one or more joints and/or muscles associated with the one or more extremities of the patient.

3-4. (canceled)

5. The method of claim 1, wherein
performing a movement evaluation of one or more extremities of the patient comprises tracking a location of one or more markers located on and/or within the patient as the one or more extremities are moved through one or more trajectories, and
determining passive movement of one or more muscles of the patient comprises determining passive movement of the one or more muscles based, at least in part, on the tracked location of the one or more markers.

6. The method of claim 5, wherein at least one of the one or more markers is located on the medical device.

7. The method of claim 5, wherein tracking a location of one or more markers comprises performing imaging of the patient as the one or more extremities are moved through the one or more trajectories to generate a plurality of images, the plurality of images including representations of at least some of the one or more markers.

8-10. (canceled)

11. The method of claim 1, wherein determining a fixation plan for fixing at least a portion of the medical device to the one or more anatomical tissues of the patient comprises determining a number of fixation points to use to fix the at least a portion of the medical device to the one or more anatomical tissues of the patient.

12. The method of claim 1, wherein determining a fixation plan for fixing at least a portion of the medical device to the one or more anatomical tissues of the patient comprises determining which of the one or more anatomical tissues of the patient to fix the at least a portion of the medical device.

13. The method of claim 1, wherein determining a fixation plan for fixing at least a portion of the medical device to the one or more anatomical tissues of the patient comprises determining a fixation plan that minimizes predicted movement of the medical device in the patient as the patient moves during usage of the medical device.

14. The method of claim 1, further comprising:
fixing the at least a portion of the medical device to the one or more anatomical tissues of the patient based on the fixation plan.

15. The method of claim 14, further comprising:
monitoring a position stability of the medical device as the patient ambulates during usage of the medical device; and
determining an updated fixation plan based, at least in part, on the monitoring.

16. The method of claim 1, wherein
the medical device is implanted in the patient, and
performing a movement evaluation of one or more extremities of a patient associated with the medical device is performed as the patient ambulates during usage of the medical device.

17. The method of claim 1, wherein the medical device is an implantable heart pump.

18. The method of claim 1, wherein
the medical device includes a catheter coupled between a first portion of the medical device configured to be implanted in the patient and a second portion of the medical device configured to be located outside of the patient, and
determining a fixation plan comprises determining a fixation plan for fixing a portion of the catheter configured to be located inside of the patient to the one or more anatomical tissues of the patient.

19. The method of claim 1, wherein the one or more anatomical tissues of the patient includes one or more of a pectoralis minor muscle, a pectoralis major muscle, a trapezius muscle, a deltoid muscle, or an external oblique muscle.

20. The method of claim 1, wherein the movement evaluation is performed after inserting the medical device into the patient and prior to operation of the medical device.

21. The method of claim 20, further comprising:
fixing the at least a portion of the medical device to the one or more anatomical tissues of the patient based on the fixation plan.

22. The method of claim 1, wherein the fixation plan is further based, at least in part, on information about how the patient may move during usage of the medical device.

23. The method of claim 22, wherein determining a fixation plan for fixing at least a portion of the medical device to one or more anatomical tissues of the patient based, at least in part, on the passive movement of the one or more muscles comprises determining a fixation plan for fixing at least a portion of the medical device to the one or more muscles.

24. (canceled)

25. The method of claim 1, wherein determining a fixation plan for fixing at least a portion of the medical device to one or more anatomical tissues of the patient based, at least in part, on the passive movement of the one or more muscles comprises determining a fixation plan for fixing at least a portion of the medical device to the one or more muscles.

26. A position support evaluation system for a medical device, the system comprising:
at least one computer processor programmed with a plurality of processor executable instructions that, when executed by the at least one computer processor perform a method, the method comprising:
determining passive movement of one or more muscles of a patient associated with the medical device during a movement evaluation of one or more extremities of the patient;
determining a fixation plan for fixing at least a portion of the medical device to one or more anatomical tissues of the patient based, at least in part, on the passive movement of the one or more muscles; and
outputting an indication of the fixation plan.

27-43. (canceled)
